(19) United States
(12) Patent Application Publication
(10) Pub. No.: US 2004/0219512 A1
(43) Pub. Date: Nov. 4, 2004

(54) Screening methods for cataractogenic risk

(75) Inventor: Christopher J. Somps, East Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/833,520

(22) Filed: Apr. 28, 2004

Related U.S. Application Data
(60) Provisional application No. 60/466,720, filed on Apr. 30, 2003.

(51) Int. Cl. C12Q 1/00

(52) U.S. Cl. 435/4

(57) ABSTRACT

The present invention relates to methods of screening candidate therapeutics for their ability to induce cataractogenesis by measuring the flux of ions through potassium ion channels.

### CROSS REFERENCE TO RELATED APPLICATION

This application claims the benefit of U.S. Provisional Application No. 60/466,720, filed on Apr. 30, 2003 and incorporated herein by reference in its entirety.

### FIELD OF THE INVENTION

The present invention relates to methods of screening for drug induced cataractogenic risk by measuring the flux of potassium ions through lens cell ion channels.

### BACKGROUND OF THE INVENTION

A cataract is the condition in which the crystalline lens of the eye becomes clouded or opaque, impairing vision and, if untreated, can lead to blindness. Through a complex series of chemical events, opacification ensues, and hence there appear to be a wide variety of potential causes of cataract.

Cataracts are a significant public health problem and result in significant cost to society. In the U.S., for example, a significant portion of Medicare budget is spent on cataract surgery.

It is known that certain chemical agents, including drug substances, can cause cataracts. Several industries, including those that produce chemicals, cosmetics and food additives, as well as the pharmaceutical industry have a primary interest to ensure that their products are safe. As such, much attention has been directed to minimizing the risk of toxicity from such products, including toxicity that might result in cataracts.

Zhang, J. J. (1994) disclose that tamoxifen blocks chloride channels on the lens and suggests that this might be a mechanism responsible for known side effects of tamoxifen, which include cataracts.

Zhang, J. J. and Jacob, T. J. C. (1996) suggest that chloride channels are involved in volume regulation of the lens and that inhibition of such channels results in lens swelling and opacification.

The use of patch clamp technology to measure potassium channel conductance in cultured lens epithelial cells has been disclosed in Cooper, K., et al. (1990).

Rae, J. L. (1994) discloses that isolated epithelial cells of chick, pig, monkey, rabbit, bovine and human lenses contain potassium channels that often turn on with a delay after a voltage step and have a larger macroscopic conductance for outward rather than inward currents.

Human lens epithelial cell lines have been disclosed in U.S. Pat. No. 5,885,832 and Ibaraki, N. (1998).

Both in vitro and in vivo methods for evaluating the cataractogenic potential of putative drugs and other agents have been disclosed. For example, typically, in vivo methods involve treating animals with a test agent, following which the lenses of the eye are tested in vivo to determine the level of cataract formation. Such methods typically involve the use of instruments such as the slit lamp microscope. Wegener, A. et al. (1989); Toogood, J. H. et al. (1993).

Cataractogenic potential of test agents may also be evaluated in vitro. Such methods include the use of explanted cultured animal lenses that are exposed to test agents and then evaluated for cataract formation. Aleo, M. D. et al. (2000); Xu, G. T. et al. (1992). Variations of such methods includes those that use of combinations of in vivo and in vitro methods.

The in vivo methods for testing cataractogenesis are advantageous over available in vitro methods in that they do not require physically removal of lenses from test animals. However, both types of methods have shortcomings. For example, with the slit lamp method which requires the projection of a two-dimensional image, it is difficult to make a sharp image due to the thickness of the lens. Jaffe, N. S. and Horwitz, J. (1992). Other disadvantages include a relatively slow rate of testing speed (low throughput) and a requirement of relatively high quantities of agents for testing.

Both the in vivo methods as well as the in vitro explant methods have the additional disadvantage of requiring the use of laboratory test animals. Although the use of animals in laboratory research has provided important contributions to the improvement of human and animal health, it is an objective of the pharmaceutical industry to replace animal experiments wherever appropriate with in vitro biological systems that can provide at least a comparable assessment of safety risk.

Although the currently available methods for evaluating ocular toxicity of test agents have served a useful and important function, there nevertheless exists a need for new in vitro methods that provide a reliable and accurate assessment of potential catarctogenicity.

### SUMMARY OF THE INVENTION

The present invention relates, in part, to methods for characterizing a test agent comprising treating a mammalian cell with a test agent; and characterizing the cataractogenic potential of the test agent by determining the effect of the agent on the flux of potassium ions through the membrane of said cell, wherein said cell comprises a potassium ion channel.

Another aspect of the present invention provides methods for characterizing a test agent comprising: treating a mammalian cell with a test agent; characterizing the cataractogenic potential of the test agent by measuring the effect of the agent on the flux of potassium ions through the membrane of said cell by an electrophysiological method, wherein said cell comprises a potassium ion channel.

A further aspect of the invention provides methods for characterizing a test agent comprising: treating a mammalian cell with a test agent; determining the effect of the agent on the flux of potassium ions through the membrane of said cell; and characterizing the test agent as one of the following: cataractogenic, provided the agent causes a change in potassium ion flux through said cell; or not cataractogenic, provided the agent does not cause a change in potassium ion flux though said cell, wherein said cell comprises a potassium ion channel.

In a preferred embodiment of the invention, the mammalian cell is a lens epithelial cell, more preferably a SRA 01/04 cell.

In another preferred embodiment, the determination of the effect of the test agent on the flux of potassium ion through using an electrophysiological method is whole-cell patch-clamp electrophysiology.

For convenience, before further description of the present invention, certain terms employed in the specification, examples, and appendant claims are collected here. These definitions should be read in light of the entire disclosure and understood as by a person of skill in the art.

The singular forms “a”, “an”, and “the” include plural references unless the context clearly dictates otherwise. “Cataractogenesis” means the induction of opacity, partial or complete, of one or both eyes, on or in the lens or capsule, especially an opacity impairing vision or causing blindness. Cataractogenesis also refers to a partial induction of opacity beneath the detection limit of conventional methods of detecting such opacity, and also refers to the processes or chemical states that induce or trigger such induction.

“Clamping” as used when referring to electrophysiological methods for the study of ion channels means the voltage or current produced across a membrane (due to ion movement) unchanged. When voltage is clamped, the method measures only current produced by the ion movement through a channel and allows a direct measurement of ionic current across a membrane.

“Conductance” means the readiness with which ions travel through a channel, measurable in siemens (S).

“Current” means the rate of ion flux across or through an ion channel.

“Electrophysiological method” means any method for measuring the flow of ions or voltage in biological tissues and, in particular, the electrical recording techniques that enable the measurement of this flow. These include so-called passive recording, as well as the “voltage clamp” and “patch-clamp” techniques, which “clamp” or maintain the cell potential at a specified level. This control may be established using feedback through an operational amplifier circuit. Control of the membrane potential is most obviously of value in the study of voltage-gated ion channels, but also aids in characterizing conductance. The most common electrophysiological recording techniques establish electrical contact with the inside of a cell or tissue with a “glass electrode.” Such an electrode is fashioned by the experimenter from a fine glass tube of about 1 mm diameter, which is then pulled to an even finer (but still hollow) tip under heat and allowed to cool. This glass “micropipette” is then filled with a chloride-based salt solution, and a chloride-coated silver wire is inserted to establish an electrochemical junction with the pipette fluid and the tissue or cell into which the pipette is inserted (typically with the aid of a microscope and finely adjustable pipette holders, known as micromanipulators). The chloride-coated silver wire connects back to the amplifier. The biological currents may be recorded on an oscilloscope, recorded onto chart paper, or recorded using a computer.

“Flux” means the flow, or movement of ions across a cell membrane. “Including” means “including but not limited to”. “Including” and “including but not limited to” may be used interchangeably.

“Induce” means to cause or produce.

“Ion” means any atom or molecule having gained or lost electrons from its normal complement of electrons, and hence carries a net negative or positive charge. Exemplary ions include, but are not limited to, potassium ions, sodium ions, hydrogen ions, calcium ions, chloride ions, hydroxide ions, sulfate ions, and phosphate ions.

“Lens epithelial cell” means any cell comprising or that formerly comprised the lens epithelium of an eye, for example, cells isolated from a lens. In addition, the term “lens epithelial cell” comprises the primary cells or progeny cells of a lens epithelial cell line culture.

“Test agent” means a chemical or biological agent whose cataractogenic potential is to be characterized.

### BRIEF DESCRIPTION OF THE DRAWINGS

FIG. 1. depicts a photomicrograph of a cultured SRA 01/04 cell under whole cell patch-clamp electrophysiology configuration.

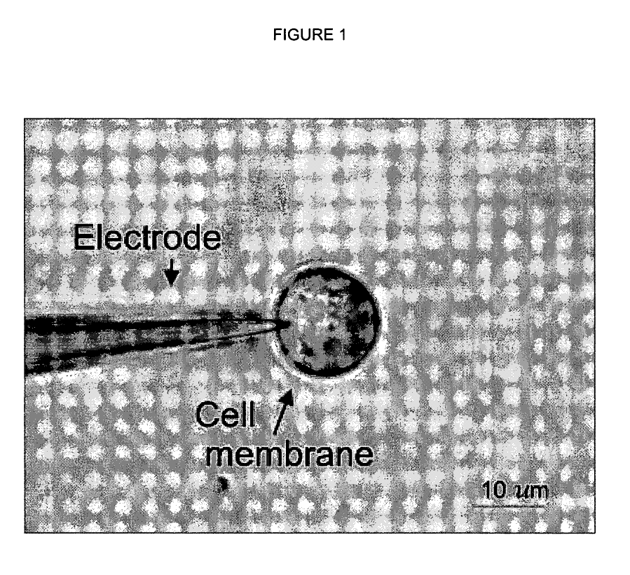

FIG. 2 depicts a schematic of a cell under whole cell patch-clamp electrophysiology configuration.

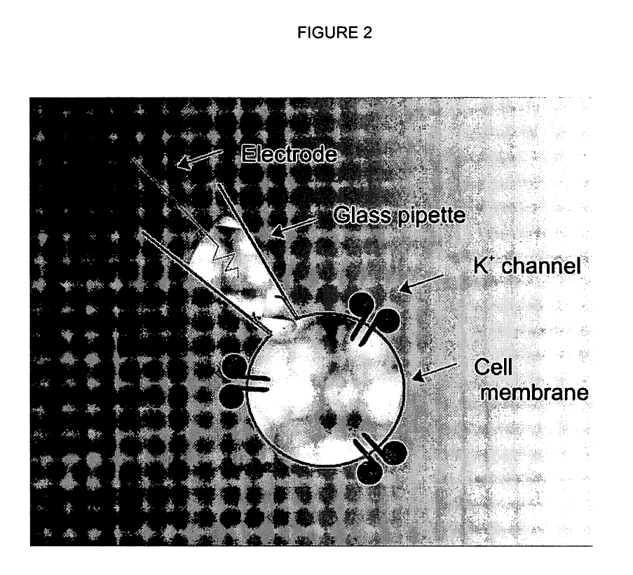

FIG. 3 depicts the waveform traces of depolarization-activated outward currents measured in a cultured SRA 01/04 cell in both the absence and presence (with concentrations noted) of TEA, a broad-spectrum potassium channel blocker.

FIG. 4 depicts dose response curves obtained from the waveform traces of FIG. 3 showing that TEA blocks the outwardly-rectified currents in a dose-dependent manner.

FIG. 5 depicts dark field photomicrographs of a rodent lens explants treated over seven days in control media and media containing 10 mM TEA.

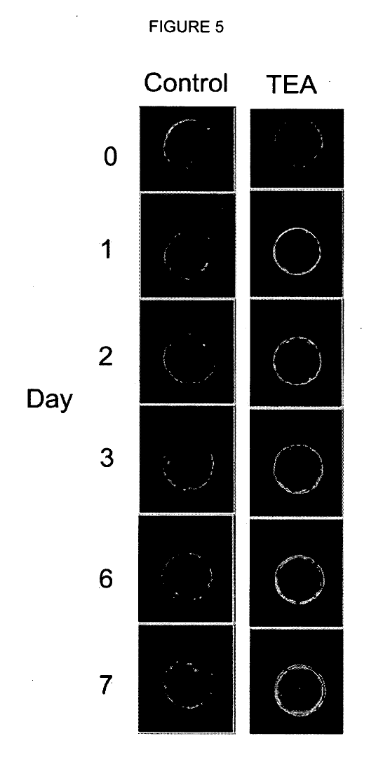

FIG. 6 depicts pixel intensity histograms from lens explants imaged after five days in culture in the absence or presence of 10 mM TEA. TEA causes a rightward shift in the intensity histogram.

### DETAILED DESCRIPTION-OF THE INVENTION

The abbreviations used herein have their usual meaning in the art. However, to even further clarify the present invention, for convenience, the meaning of certain abbreviations are provided as follows: “° C.” means degrees centigrade; “μl” means microliter; “ATCC” means the American Type Tissue Culture Collection located in Manassas, VA (website at www.atcc.org); “cDNA” means complementary DNA; “dL” means deciliter; “DMEM” means Dulbecco's modified Eagle's medium; “DMSO” means dimethyl sulfoxide; “DNA” means deoxyribonucleic acid; “EDTA” means ethylenediamine tetra-acetic acid; “EGTA” means ethylene glycol-bis(b-aminoethylether)-N,N,N′,N′-tetraacetic acid; “EMEM” means Eagle's minimum essential medium; “FBS” means fetal bovine serum; “g” means gram; “GΩ” means gigaohm; “HEPES” means N-2-hydroxyethylpiperazine-N′-2-ethanesulfonic acid; “kg” means kilogram; “NaCl” means sodium chloride; “NaOH” means sodium hydroxide; “MEM” means Modified Eagle's Medium; “mRNA” means messenger RNA; “mg” means milligram; “mL” means milliliter; “mM” means millimolar; “MΩ” means megaohm; “mOsm” means milliosmol; “ms” means millisecond; “ng” means nanogram; “Ω” means ohm “PCR” means polymerase chain reaction; “PBS” means phosphate buffered saline; “RNA” means ribonucleic acid; “RPM” means revolutions per minute; and “RT-PCR” means reverse transcriptase polymerase chain reaction; “TEA” means tetraethylammonium; “xg” means centrifugal force measured as number of gravities.

The mammalian lens is an avascular tissue that comprises precisely packed multiple layers of cells containing high concentrations of lens proteins (predominantly the alpha and gamma crystallins). Transparency of the eye lens is maintained by ordered packing of highly concentrated lens protein in the fiber cells via short-range interactions to form a dense glasslike liquid phase. Epithelial cells are present in a single layer on the anterior surface of the lens, and are responsible for the metabolic activity, maintenance of ion gradients, and synthesis of proteins responsible for the function of the lens. After epithelial cells proliferate on the anterior layer, they undergo terminal differentiation into elongated fiber cells as they migrate toward the center of the lens. Old fiber cells are not removed from the lens; they are simply compressed into the center of the lens as younger fiber cells are laid down over them. As the fiber cells mature and are compressed, they lose their nuclei and other organelles, become dehydrated, cease protein turnover and, by the time they are about one-third of the way into the lens, lose nearly all of their metabolic activity. These mature fiber cells can be thought of as “inert bags of concentrated protein solution” which can have ultimate protein concentrations of up to 700 mg/mL in the very center of the lens.

Because of the absence of blood vessels in the lens, the lens relies on the epithelial cells to take up solutes and ions from the aqueous humor. The epithelial cells must then provide all the necessary metabolic requirements to the fiber cells in the lens. To accomplish its unique role, the lens uses a vast array of channels, pumps, transporters and intercellular connections at gap junctions to distribute nutrients, remove metabolites, and maintain the proper ionic balance and hydration for both epithelial and lens fiber cells.

This invention is based, in part, upon the discovery that the cataractogenic potential of a test agent can be predicted by measuring the compound's ability to block potassium ion channel conductance in the lens of the eye. It has been found that compounds which block potassium channels are likely to cause lens cataracts. This invention provides a simple screening method for identifying cataractogenic agents.

Without wishing to be bound by any particular theory as to the relevant mechanism of action, it is believed that cataract formation is connected to the regulation of lens epithelial cell volume. It is further believed that the swelling of the cells causes changes in the cells' internal environment which, in turn, result in disruption to internal subcellular components. The effect of such disruption is a loss in lens clarity. Most importantly, it is believed that potassium channels are a key factor in the regulation of cell volume and that blockage of such channels results in cell swelling and, ultimately, cataracts.

### A. Cells

In one embodiment of the invention, an agent is characterized for its cataractogenic potential by treating a cell with the agent and measuring the effect of such treatment on potassium ion current of the cell. Any mammalian cell that expresses potassium ion channels on its surface may be used for the practice of this embodiment of the invention. Such cells may include mammalian cells that express the potassium ion channel endogenously as well as cells that have been genetically modified to express potassium ion channels. In a preferred embodiment, such cells are derived from mammalian lens epithelial cells.

Any type of mammalian lens epithelial cell may be used in the methods of this invention, including epithelial cells obtained directly from mammalian lens tissue explants and epithelial cells derived from lens mammalian epithelial cell lines. Those with skill in the art will appreciate that, when predicting cataractogenic risk in a particular mammalian species, for example, human, by the methods of this invention it is preferable to use epithelial cells derived from that same species.

1. Preparation of Primary Cultures of Lens Epithelial Cells

Primary cell cultures may be initiated from the anterior lens capsule and its attached epithelium by methods known to those with skill in the art, based upon the present disclosure. For example, lens capsule and their adhering epithelia may be obtained by careful microdissection from autopsied ocular globes, preferably within no more than six hours after death, or from ophthalmic surgery, for example from cataract or vitreous surgery. A preferred source is lens epithelium of infants obtained in vitreous surgery or for the treatment of retinopathy of prematurity.

For each capsule, all excess capsule and fiber cells are trimmed away leaving only the monolayer of the epithelium and its capsule. The epithelia and capsules are then incubated at room temperature in a solution of 0.125% collagenase (Type IV, Worthington Biochemical Corp., Lakewood, N.J.) and 0.05% protease (Type XXIV, Sigma-Aldrich Co., St. Louis, Mo.) in low calcium, sodium aspartate Ringer at pH 7.35. After for one and one-half to two hours, the capsules with epithelial cells attached are then gently removed from the enzyme solution and placed in five milliliters of a standard sodium chloride Ringer solution (149.2 mM NaCl, 4.74 mM KCL, 2.54 mM CaCl₂, 5.0 mM Hepes and 10 mM glucose (pH 7.35 and osmolality 305 mOsm/l). The cells are dissociated from the capsule by gentle trituration with a fire-polished Pasteur pipette and the resulting suspended cells are spun down by centrifugation at 350×g for 3-5 minutes. The cells are then resuspended in fresh standard sodium chloride Ringer solution.

2. Preparation of Lens Epithelial Cell Lines

Although human donor lens epithelial cells are available, the supply is nevertheless scarce. Moreover, lens epithelial cells obtained from adult or senile cataract patients are either incapable of growth in cell culture or cannot be subcultured and those from infants have limited proliferative potency and degenerate over time. Ibaraki, N. et. al. (1998). For these reasons, cells derived from lens epithelial cell lines are preferred in the practice of the methods of this invention.

Mammalian lens epithelial cell lines may be prepared by methods known to those with skill in the art, based upon the present disclosure. For example, cells isolated by the method described above for preparing primary cultures are suspended in a liquid medium containing 10-20% fetal bovine serum or calf serum, such as EMEM, Dulbecco's modified EMEM, HAM medium F12 or Katsuta medium DM-160, and preincubated in a carbon dioxide incubator for 14 to 21 days. The resulting cells are then immortalized by methods known to those with skill in the art, based upon the present disclosure. For example, the cells may be infected with a virus, such as the simian virus 40 (SV40) (see Andley, U. P. et al. (1994)) that imparts immortality through the SV40 large T antigen gene. More preferably, the cells are transfected by method known to those with skill in the art with a plasmid containing the SV40 large T antigen gene (Genbank accession no. VIRU0033) (see Ibaraki, et al. (1998)).

The following publications describe various methods of creating and culturing lens epithelial cell lines, and the contents of each are hereby incorporated by reference in their entireties: Bermbach, G., et al. (1991); Chandrasekharam, N. N. and Bhat, S. P. (1989); Jacob, T. J. C. (1987); Lipman, R. D. and Taylor, A. (1987); Reddan, J. R., et al. (1982/1983). Also, lens epithelial cell lines are commercially available, for example, the human lens epithelial cell line CRL-11421 from ATCC and FERM BP-5454 from the National Institute of Bioscience and Human-Technology Agency of Industrial Science and Technology, Ibaraki, Japan.

In a preferred embodiment, lens epithelial cells used in the methods of the invention are the human lens epithelial cell line SRA 01/04. The cell line SRA 01/04 is available from the National Institute of Bioscience and Human-Technology Agency of Industrial Science and Technology, Ibaraki, Japan, under international deposit No. FERM BP-5454.

3. Preparation of Mammalian Cells that Express Potassium Channels

The manner of genetically modifying a mammalian cell to express a potassium ion channel will be apparent to those with skill in the art based upon the present disclosure. For example, methods for transfecting potassium channels into Chinese hamster ovary (CHO) and HEK-293 cells are described in Shepard, A. R. and Rae, J. L. (1999).

Generally, to express a biologically active potassium ion channel, a suitable nucleotide sequence encoding a potassium channel is inserted into an appropriate expression vector. A number of potassium ion channel sequences are known in the art, such as, for example, Kv2.1 (Genbank accession no. AF026005), described in Rae, J. L. and Shepard, A. R. (1998) and Kv6.3 (Genbank accession no. AB070604), described in Sano, Y. (2002). Others will be apparent to those with skill in the art based upon the present disclosure.

Based upon the present disclosure, those with skill in the art may use any suitable known method to construct expression vectors containing sequences encoding a potassium ion channel and appropriate transcriptional and translational control elements. These methods include, for example, in vitro recombinant DNA techniques, synthetic techniques, and in vivo genetic recombination (see, e.g., Sambrook, J. et al. (1989); Ausubel, F. M. et al. (2001)).

The elements for transcriptional and translational control in a suitable host of the inserted coding sequence of a potassium ion channel may include regulatory sequences, such as enhancers, constitutive and inducible promoters, and 5′ and 3′ untranslated regions in the vector and in polynucleotide sequences encoding a potassium channel. As those skilled in the art will appreciate, such elements vary in their strength and specificity. Specific initiation signals may also be used to achieve more efficient translation of sequences encoding a potassium channel. Such signals include the ATG initiation codon and adjacent sequences, e.g. the Kozak sequence. For the purpose of carrying out the invention where sequences encoding a potassium ion channel and its initiation codon and upstream regulatory sequences are inserted into the appropriate expression vector, no additional transcriptional or translational control signals may be needed. However, where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (see, e.g., Scharf, D. et al. (1994)).

It will further be appreciated by those with skill in the art, based upon the present disclosure, that any suitable selection systems (“selectivity markers”) may be used to recover transformed cell lines. These include, but are not limited to, the herpes simplex virus thymidine kinase and adenine phosphoribosyltransferase genes, for use in tk- and apr- cells, respectively (see, e.g., Wigler, M. et al. (1977); Lowy, I. et al. (1980). Antimetabolite, antibiotic, or herbicide resistance can also be used as a basis for selection. For example, dhfr (dihydrofolate reductase) confers resistance to methotrexate; neo confers resistance to the aminoglycosides neomycin and G-418; and als and pat confer resistance to chlorsulfuron and phosphinotricin acetyltransferase, respectively (see, Wigler, M. et al. (1980); Colbere-Garapin, F. et al. (1981)). Additional selectable genes have been described, e.g., trpB and hisD, which alter cellular requirements for metabolites (see, e.g., Hartman, S. C., et al. (1988)). Visible selectivity markers, e.g., anthocyanins, green fluorescent proteins (Clontech, Palo Alto, Calif.), β glucuronidase and its substrate β-glucuronide, or a luciferase and its substrate luciferin may be used. These markers can be used not only to identify transformants, but also to quantify the amount of transient or stable protein expression attributable to a specific vector system (see, e.g., Rhodes, C. A. (1995)).

Although the presence or absence of selectivity marker gene expression suggests, as the case may be, that the gene of interest is also present, it may be desirable, in any such case, to confirm the presence and expression of the gene. For example, if the sequence encoding the potassium channel is inserted within a selectivity marker gene sequence, transformed cells containing sequences encoding a potassium channel can be identified by the absence of selectivity marker gene function. Alternatively, a selectivity marker gene can be placed in tandem with a sequence encoding a potassium channel under the control of a single promoter. As those skilled in the art will appreciate, based upon the present disclosure, expression of the marker gene in response to induction or selection generally indicates expression of the tandem gene as well.

Host cells that contain a nucleotide sequence encoding a potassium channel may be identified by a variety of procedures known to those of skill in the art based upon the present disclosure. These procedures include, but are not limited to, DNA-DNA or DNA-RNA hybridizations, PCR amplification, and protein bioassay or immunoassay techniques which include membrane-, solution- and/or chip-based technologies for the detection and/or quantification of nucleotide or amino acid sequences. Such methods and techniques are described, for example, in Ausubel, F. M. et al. (2001).

### B. Methods of Measuring Potassium Ion Flux

Any of a variety of techniques known by those with skill in the art for measuring the flux of ions in a lens cell may be used with the present invention, including, but not limited to, electrophysiological techniques and methods of imaging ion flux such as the use of fluorescent dyes. However, the preferred method employs electrophysiological techniques.

As those with skill in the art will appreciate, based upon the present disclosure, any electrophysiological technique which can measure electrical conductances in a cell may be used in such methods. Examples include intracellular microelectrode recording (indirect measurement), two microelectrode voltage clamp, and single microelectrode voltage clamp.

Patch-clamp techniques and improvements thereof, have been developed to study electrical currents in cells, in particular to study ion transfer across a cell membrane, and in some cases the current through a single channel in a cell. To measure these currents, the membrane of the cell is generally closely attached to the opening of the patch micropipette so that a very tight seal is achieved. This seal prevents current from leaking outside of the patch micropipette. The resulting high electrical resistance across the seal can be exploited to apply voltages and perform high resolution current measurements and apply voltages across the membrane. Different configurations of the patch-clamp technique may be used. (Sakmann, B. and Neker, E. (1984). For the present invention, the cell-attached mode of the patch-clamp technique can be used to record the existence of potassium channels and the inside-out and outside-out patch configurations may be used to record the sensitivity of potassium channels to various chemicals. Cooper, K. et al. (1990) describe the use of patch-clamp technology to measure potassium channel conductance in cultured lens epithelial cells.

In general, a patch-clamp measurement may be performed as follows, with suitable adaptations to the technique made depending on the ion to be measured and the type of cell. A glass microcapillary or micropipette is filled with a saline buffer solution and fitted with a microelectrode. The function of the electrode is to provide an electrical connection to a wire via the reversible exchange of ions in the pipette solution. Through the use of a microscope and micromanipulating arm, the user finds a biological cell or cell membrane containing ion channels of interest and gently touches the cell membrane with the pipette. The measurement circuit is completed via the external ionic solution and a second bath electrode. A high-impedance operational amplifier senses the current flowing in the circuit which is subsequently recorded and analyzed with a data recording system. The key to the function of the technique is the ability to form a high electrical resistance (about 1 gigaohm) seal between the glass pipette and the cell membrane, so that the current recorded by the amplifier is dominated by ions flowing through the cell membrane and not ions flowing around the glass pipette directly into the bath solution.

One of the more common measurement configuration (although many are possible) is the whole cell voltage clamp. In this configuration it is necessary to permeabilize the portion of membrane at the end of the pipette so as to effectively place the pipette electrode inside the cell. This, in turn, allows for an external voltage command to be placed between the intracellular pipette electrode and the extracellular bath electrode, thereby providing control of the cell's transmembrane voltage potential. The term “whole cell” is derived from the fact that with this configuration, the instrument measures the majority of the currents in the entire cell membrane.

The electrical permeabilization of the membrane at the end of the pipette can be induced in many ways but is often achieved by voltage pulses of sufficient strength and duration such that the membrane inside the pipette physically breaks down. This is commonly referred to as “zapping” and is a well-known technique in the field. Another technique utilized to electrically permeabilize the membrane is through the use of certain antibiotics such as Nystatin and Amphotericin B. These chemicals work by forming chemical pores in the cell membrane that are permeable to monovalent ions such as chloride. Since chloride is the current carrying ion for the commonly used Ag/AgCl electrode, these antibiotics can produce a low resistance electrical access to the interior of the cell. The advantage of the chemical technique is that the membrane patch remains intact such that larger intracellular molecules remain inside the cell and are not flushed out by the pipette solution as with the zapping technique. The use of chemicals to electrically permeabilize the membrane is also a commonly used technique in the field and is referred to as a “perforated patch”.

The formation of the high-resistance electrical seal enables the measurement system to detect very small physiological membrane currents, (e.g., 10⁻¹² amp). In addition, by perforating a portion of the cell membrane either electrically or chemically, it is possible to control the voltage (voltage clamp) or current (current clamp) across the remaining intact portion of the cell membrane. This greatly enhances the utility of the technique for making physiological measurements of ion channel/transporter activity since quite often this activity is transmembrane voltage dependent. By being able to control the trans-membrane voltage (or current), it is possible to stimulate or deactivate ion channels or transporters with great precision and as such greatly enhance the ability to study complex drug interactions.

U.S. Pat. No. 6,488,829 describes a device whereby electrophysiological measurements can be made on cells or cell membranes in a manner which allows for multiple measurements to be made in parallel, without direct human intervention.

Various descriptions of other adaptations of the above-described patch-clamp technique are described in the following patents, patent applications, and publications and are hereby incorporated by reference in their entireties: Denyer, J., et al. (1998); Ausubel, F. M., et al. (2001); Neher E., et al. (1976); Neher E., et al. (1978); Hammill, O. P., et al. (1981); Sherman-Gold, R. (1993); Rae, J., et al. (1991); and Sakmann, B. and Neher, E. (1995); U.S. Pat. No. 6,063,260 to Olesen, et al.; and PCT application publication number WO 99/66329.

As those with skill in the art will appreciate based upon the present invention, methods that are not based upon electrophysiological measurements may also be used for measuring ion flux in the practice of this invention. For example, potassium flux may be measured, using radioactive potassium surrogate ions such as ⁸⁶rubidium (⁸⁶Rb+). Diecke et al. (1998); and, Diecke and Beyer-Mears (1997).

In a preferred embodiment of the methods of the invention with particular utility to the pharmaceutical industry for drug discovery, the methods may be adapted to systems that are automated. Automation of these methods provides the capability of screening a high number of test agents within a relatively short period of time and the additional advantage of requiring relatively small quantities of test agent. Automated systems based upon patch-clamp electrophysiological techniques are available commercially. Exemplary systems include the lonWorks™ HT system (Molecular Devices Corporation, Sunnyvale, Calif.) and the PatchXpress™ 7000A Automated Parallel Patch-Clamp System (Axon Instruments, Union City, Calif.).

In other embodiments, fluorescence microscopy may be used to measure single ion channel flux. For example, calcium ion influx through individual N-type calcium ion channels may be measured using confocal microscopy, or confocal fluorescence imaging. Single channel calcium transients (SCCATs) as brief as 10 ms may be resolved, and channel lifetime and latency distributions may be measured. Confocal fluorescence imaging provides temporal information on channel gating similar to that obtained by patch-clamp recording. Moreover, optical imaging provides spatial information from multiple channels, involves minimal disruption and is applicable to channels that are not accessible to a patch pipette.

Assays and methods of developing assays appropriate for use in the methods described above are known to those of skill in the art, and are contemplated for use as appropriate with the methods of the present invention. All of the above screening methods may be accomplished using a variety of assay formats. Assay formats which approximate such conditions as hypo-osmolarity or dosage of a candidate therapeutic, may be generated in many different forms. I

The disclosures of all patents, applications, publications and documents, including brochures and technical bulletins, cited herein, are hereby expressly incorporated by reference in their entirety. It is believed that one skilled in the art can, based on the present description, including the examples, drawings, and attendant claims, utilize the present invention to its fullest extent.

The following Examples are to be construed as merely illustrative of the practice of the invention and not limitative of the remainder of the disclosure in any manner whatsoever.

### EXAMPLES

### Example 1

### The Effect of TEA On Lens Cell Current Using Whole-Cell Patch-Clamp Recording Method

Preparation of Lens Epithelial Cell Line Culture Cells from the cell line, SRA 01/04 (deposited in the National Institute of Bioscience and Human-Technology Agency of Industrial Science and Technology, Ibaraki, Japan, international deposit No. FERM BP-5454) were thawed and plated on the bottom of a 25 square centimeter tissue culture flask (Falcon™ Flask, BD Biosciences, Lincoln Park, N.J.) and grown in high-glucose DMEM supplemented with 15% of FBS (Invitrogen, Grand Island, N.Y.) and 1% of penicillin/streptomysin (Invitrogen) at 37° C. in 5% CO₂ and 95% air environment. The medium was changed twice a week. When confluence of 90-100% was reached (about 7-14 days), the medium was removed from the culture flask and the cells on the bottoms were washed with one ml of trypsin-EDTA (0.05% trypsin and EDTA, Invitrogen). The cells were then treated with one ml of trypsin-EDTA solution in an incubator at 37° C. for three and one-half to five minutes. The trypsin was then inactivated by adding four to eight ml of DMEM containing 15% FBS and centrifuging the resulting cell suspension at 1000×g for four to five minutes at room temperature. The SRA 01/04 cells were then resuspended in one to one and one-half ml bath solution containing 149 mM NaCl, 4.7 mM KCI, 2.5 mM CaCl₂, 5 mM Glucose and 5 mM HEPES (pH 7.3, adjusted with NaOH) with 0.1% albumin (Sigma-Aldrich Co.) added.

Measurement of Potassium Ion Flux

The SRA 01/04 cells suspension from Example 1 was vortexed to ensure uniform suspension. Approximately 15 μl of the cell suspension was pipetted onto a glass coverslip (Warner Instruments, Hamden, Conn., USA) placed on the transparent bottoms of a recording chamber mounted on an inverted differential interference contrast microscope (Eclipse TE300, Nikon, Tokyo, Japan). Whole cell currents were measured under the whole-cell configuration at room temperature (24-26° C.) using a multiclamp 700A dual channel multi-purpose microelectrode amplifier (Axon Instruments, Foster City, Calif., USA). Electrodes were fabricated from 1.65 mm capillary glass (PG52165-4, World Precision Instruments, Sarasota, Fla.), using a Sutter P-97 microelectrode puller (Sutter Instrument Co., Novato, Calif.). Pipette tips were polished by a MF-830 microforge (Narishige International USA, Inc., East Meadow, N.Y.). Pipettes were filled with pipette solution containing 130 mM KCI, 2 mM MgATP, 5 mM MgCl₂, 10 mM HEPES and 5 mM EGTA (pH 7.3 adjusted with KOH). The bath solution used contained 149 mM NaCl, 4.7 mM KCI, 2.5 mM CaCl₂, 5 mM Glucose and 5 mM HEPES (pH 7.3, adjusted with NaOH). The resistance of solution filled pipettes was 2.5-5.0 MΩ. The osmolarity of the pipette and bathing solution was adjusted to 295 and 300 mosM/kg using a Wescor 5520 osmometer (Wescor, Inc., Logan, Utah).

After attaining the whole-cell configuration with seal resistance of greater than 1 GΩ, the series resistance was less than 10 MΩ and was compensated for by 3-30% to minimize voltage errors. Capacitance artifact was automatically canceled using the computer-controlled circuitry of the patch-clamp amplifier. Pulse generation and data acquisition were done on-line using an A/D and D/A interface, Digidata 1322A High Speed Data Acquisition (Axon Instruments). Current signals were filtered at 5 KHz and sampled at 20 kHz. The holding potential was set at −60 mV to inactivate voltage-gated Na+ and Ca²⁺ channels. To obtain the current-voltage (I-V) relationship, a series of successive step pulses of 250 ms in duration were applied from −120 to +140 mV in 20 mV increments.

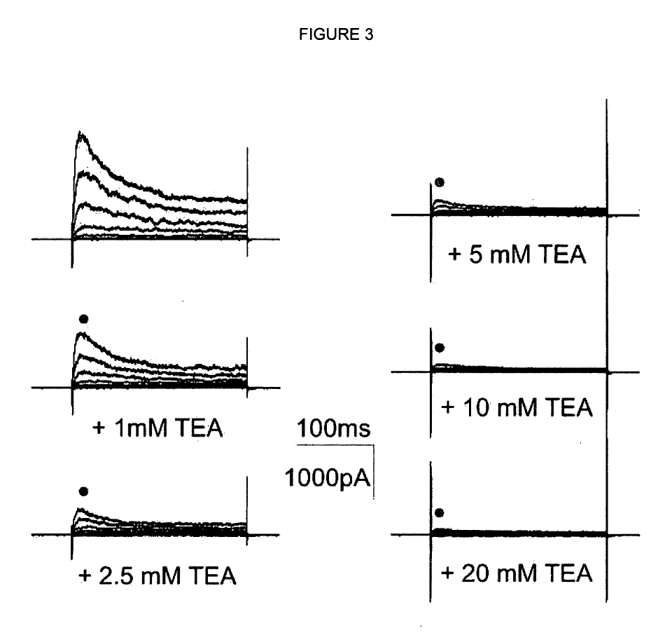

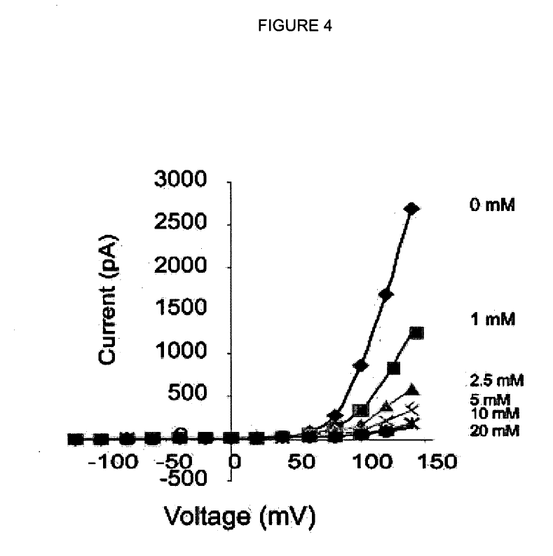

TEA was introduced directly into the bath solution by pipette at concentrations of 1, 2.5, 5, 10 and 20 mM. As indicated by FIG. 3, outwardly-rectified currents are blocked by TEA. As indicated by FIG. 4 showing the current-voltage relation in a dose-dependent manner, concentrations of TEA of greater than five mM produce no significant additional current block. These results suggest that the predominant current in lens epithelial cells may be an outwardly rectified, voltage-dependent potassium current.

Example 1 illustrates the methods of this invention for characterizing the cataractogenic potential of a test agent by measuring the effect of the agent on the flux of potassium ions though a cell membrane.

### Example 2

### Effect of TEA on Rodent Lens Explants

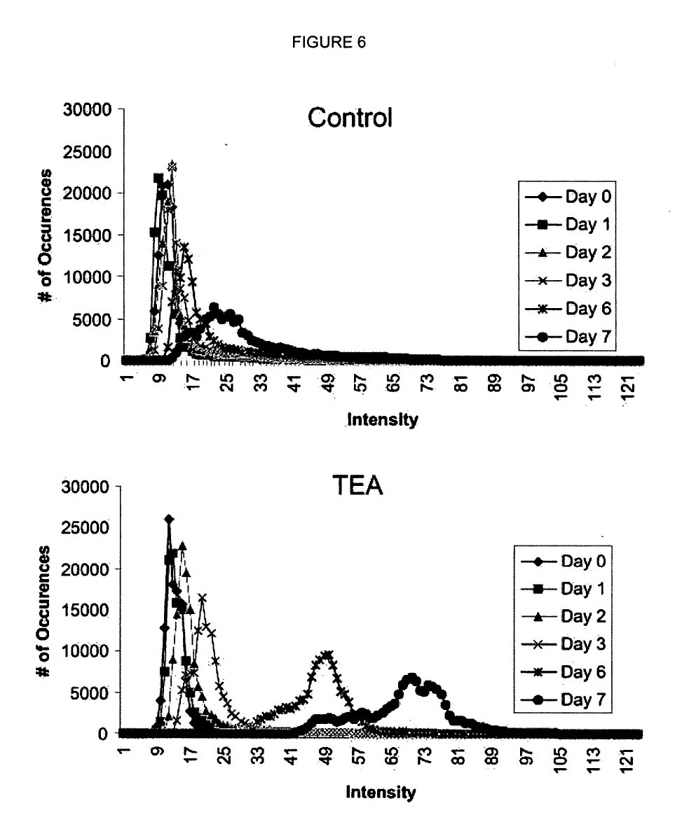

Rodent lenses were surgically removed and placed in tissue culture 199 (TC199) media (Invitrogen) supplemented with bicarbonate buffer with and without 10 mM TEA (Sigma Aldrich Co.). Darkfield photomicrographs of the cells were taken over a seven day period. As indicated by FIGS. 6 and 7, treatment with TEA causes increased clouding of the lens as compared with the control.

Example 2 illustrates the cataract causing potential of a known potassium ion channel blocking agent.

### Equivalents

The present invention provides in part, methods of screening for cataractogenic risk by measuring the flux of potassium ions through lens cell ion channels. While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

All publications and patents mentioned herein, including those items listed below, are hereby incorporated by reference in their entireties as if each individual publication or patent was specifically and individually indicated to be incorporated by reference. In case of conflict, the present application, including any definitions herein, will control.

### References

Aleo, M. D., Avery, M. J. Beierschmitt, W. P., Drupa, C. A. Fortner, J. H., Kaplan, A. H., Navetta, K. A., Shepard, R. M. and Walsh C. M. (2000) The use of explant lens culture to assess cataractogenic potential, Annals of the New York Academy of Sciences 919, 171-87; Andley, U. P., Rhim, J. S., Chylack, L. T. and Fleming T. P. (1994) Propagation and immortalization of human lens epithelial cells in culture. Invest Opthalmol. Vis. Sci., 35, 3094-3102; Ausubel, F. M. et al. (2001) Current Protocols in Molecular Biology, John Wiley & Sons, New York, N.Y.; Bermbach, G., et al. (1991) Experimental Eye Research, 52, 113-119; Chandrasekharam, N. N. and Bhat, S. P. (1989) Current Eye Research, 8, 285-291; Colbere-Garapin, F. et al. (1981) J. Mol. Biol. 150, 1-14; Cooper, K., Gates, P., Rae, J. L. and Dewey, J. (1990) Electrophysiology of Cultured Human Lens Epithelial Cells. J. Membrane Biol 117, 285-198; Denyer, J., et al. (1998) Drug Discovery Today, 3, 323-332; Diecke, F. P. et al. (1998) Invest. Opthalmol. Vis. Sci., 39, 104-110; Diecke, F. P. and Beyer-Mears, A. (1997) Current Eye Research, 16, 279-88; Hammill O. P., et al., (1981) Pflugers Arch 391, 85-100; Hartman, S. C., et al. (1988) Proc. Natl. Acad. Sci. USA 85, 8047-8051; Hockwin, O., Sasaki, K. and Lerman, S. (1990) “Evaluating cataract development with the Scheimpflug camera,” in Noninvasive Diagnostic Techniques in Ophthalmology, B. R. Masters, ed. (Springer-Verlag, N.Y.), 282-318; Ibaraki, N., Chen, S. -C., Lin, L. -R., Okamato, H., Pipas, J. M. and Reddy, V. N. (1998) Human Lens Epithelial Cell Line. Exp. Eye Res., 67, 577-585; Jacob, T. J. C. (1987) Experimental Eye Research, 45, 93-104; Jaffe, N. S. and Horwitz, J. (1992) Lens and Cataract, Textbook of Ophthalmology, S. M. Podos, M. Yanoff, eds., Gower Medical Publishing, New York, Vol. 3; Lipman, R. D. and Taylor, A. (1987) Current Eye Research, 6, 145-157; Lowy, I. et al. (1980) Cell 22, 817-823; Neher E., et al. (1976) Nature 260, 799-802; Neher E., et al. (1978) Pflugers Arch 375, 219-228; Rae, J. L., et al. (1991) Journal of Neuroscience Methods, 37, 15-26; Rae, J. L. (1994) Outwardly Rectifying Potassium Currents in Lens Epithelial Cell Membranes. Curr. Eye Res. 13, 679-686; Rae, J. L. and Shepard, A. R. (1998) “Identification of potassium channels in human lens epithelium”, The Eye's Aqueous Humor: From Secretion To Glaucoma, Civan, M. M. (Ed.), Academic Press, San Diego, Calif., 69-104; Reddan, J. R., et al. (1982/1983) Current Eye Research, 2, 399-405; Rhodes, C. A. (1995) Methods Mol. Biol. 55, 121-131; Sakmann B. and Neher E. (1995) Single-Channel Recording, 2nd Ed., Plenum Press, New York; Sakmann, B. and Neker, E. (1984) Annual Review of Physiology, 46, 455; Sambrook, J. et al. (1989) Molecular Cloning, A Laboratory Manual, Cold Spring Harbor Press, Plainview, N.Y.; Sano, Y., Mochizuki, S., Miyake, A., Kitada, C., Inamura, K., Yokoi, H., Nozawa, K., Matsushime, H. and Furuichi, K. (2002) Molecular Cloning And Characterization of Kv6.3, a Novel Modulatory Subunit For Voltage-Gated K+ Channel Kv2.1, FEBS Lett. 513, 230-234; Scharf, D. et al. (1994) Results Probl. Cell Differ. 20, 125-162; Shepard, A. R. and Rae, J. L. (1999) Electrically silent potassium channel subunits from human lens epithelium, Am. J. Physiol. 277 (Cell Physiol. 46): C412-C424; Sherman-Gold, Rivka (ed.) (1993) The Axon Guide for Electrophysiology & Biophysics; Toogood, J. H. Markov, A. E. Baskerville J. and Dyson C. (1993) Association of ocular cataracts with inhaled and oral steroid therapy during long-term treatment of asthma. Journal of Allergy & Clinical Immunology. 91(2):571-579; Wegener A., Maierhofer O., Heints M. and Hockwin 0. (1989) Testing a possible cocataractogenic potential of befunolol (Glauconex) with animal cataract models. Journal of Ocular Pharmacology. 5, 45-50; Wigler, M. et al. (1977) Cell 11, 223-232; Wigler, M. et al. (1980) Proc. Natl. Acad. Sci. USA 77, 3567-3570; Xu, G. T., Zigler, J. S. and Lou, M. F. (1992) Establishment of a naphthalene cataract model in vitro, Experimental Eye Research 54, 73-81; Zhang, J. J. and Jacob, T. J. C. (1996) Volume Regulation in the Bovine Lens and Cataract. J. Clin. Invest 97, 971-978; Zhang, J. J., Jacob, T. J. C., Valverde, M. A., Hardy, S. P., Mintenig, G. M., Sepulvda, F. V., Gill, D. R., Hyde, S. C., Trezise, A. E. O. and Higgins, C. F. (1994) Tamoxifen Blocks Chloride Channels. J. Clin. Invest. 94, 1690-1697.

1. A method for characterizing a test agent comprising: treating a mammalian cell with a test agent; and characterizing the cataractogenic potential of the test agent by determining the effect of the agent on the flux of potassium ions through the membrane of said cell, wherein said cell comprises a potassium ion channel.

2. A method of claim 1 wherein said mammalian cell is a lens epithelial cell.

3. A method of claim 1 wherein said mammalian cell is a SRA 01/04 cell.

4. A method for characterizing a test agent comprising: treating a mammalian cell with a test agent; and characterizing the cataractogenic potential of the test agent by determining the effect of the agent on the flux of potassium ions through the membrane of said cell by an electrophysiological method, wherein said cell comprises a potassium ion channel.

5. A method of claim 4 wherein said electrophysiological method is whole-cell patch-clamp electrophysiology.

6. A method of claim 4 wherein said mammalian cell is a lens epithelial cell.

7. A method of claim 4 wherein said mammalian cell is a SRA 01/04 cell.

8. A method of claim 4 wherein said electrophysiological method is whole-cell patch-clamp electrophysiology and said cell is a lens epithelial cell.

9. A method for characterizing a test agent comprising: treating a mammalian cell with a test agent; determining the effect of the agent on the flux of potassium ions through the membrane of said cell; and characterizing the test agent as either one of the following: cataractogenic, provided the agent causes a change in potassium ion flux through said cell; or not cataractogenic, provided the agent does not cause a change in potassium ion flux though said cell, wherein said cell comprises a potassium ion channel.

10. A method of claim 9 wherein said method for determining the effect of the agent on the flux of potassium ions through the membrane of said cell, comprises an electrophysiological method

11. A method of claim 10 wherein said electrophysiological method is whole-cell patch-clamp electrophysiology.

12. A method of claim 9 wherein said mammalian cell is a lens epithelial cell.

13. A method of claim 9 wherein said mammalian cell is a SRA 01/04 cell.

14. A method of claim 10 wherein said electrophysiological method is whole-cell patch-clamp electrophysiology and said cell is a lens epithelial cell.
